(19) United States
(12) Patent Application Publication
(10) Pub. No.: US 2023/0173004 A1
(43) Pub. Date: Jun. 8, 2023

(54) Method and Composition for Treating Ringworm on Biotic and Abiotic Surfaces

(71) Applicant: Jeneil Biosurfactant Company, LLC, Saukville, WI (US)

(72) Inventor: Gary A. Strobel, Bozeman, MT (US)

(21) Appl. No.: 18/052,088

(22) Filed: Nov. 2, 2022

Related U.S. Application Data
(60) Provisional application No. 63/274,940, filed on Nov. 2, 2021.

(51) Int. Cl. A61K 36/062; A01N 63/30; A61K 31/11; A01N 35/02; A61K 31/22; A01N 37/02; A61K 31/121; A61K 31/045; A01N 31/02; A61K 31/19; A61P 31/10; A01P 3/00; A01N 25/02; A01N 37/10; A61K 31/216; A61K 9/06

(52) U.S. Cl. CPC A61K 36/062; A01N 63/30; A61K 31/11; A01N 35/02; A61K 31/22; A01N 37/02; A61K 31/121; A61K 31/045; A01N 31/02; A61K 31/19; A61P 31/10; A01P 3/00; A01N 25/02; A01N 37/10; A61K 31/216; A61K 9/06

(57) ABSTRACT

Embodiments of the disclosure relate to treatments for ringworm on biotic and abiotic surfaces. According to one aspect, a concentrated antimicrobial solution is provided. The concentrated solution includes an antimicrobial composition, a solubilizing agent, and an essential oil. The concentrated antimicrobial solution can be mixed with water, and the resulting solution can be used to disinfect abiotic surfaces from ringworm. According to another aspect, an antimicrobial cream is provided. The antimicrobial cream includes a carrier including a humectant and an antimicrobial composition dispersed in the carrier at 1% w/v or greater.

### CROSS-REFERENCE TO RELATED PATENT APPLICATIONS

This application claims priority to U.S. Provisional Application No. 63/274,940, filed Nov. 2, 2021, which is incorporated herein by reference in its entirety.

### FIELD OF THE INVENTION

This invention generally relates to an antimicrobial composition and, in particular, to a composition configured to treat ringworm infections.

### BACKGROUND OF THE INVENTION

Trichophyton is a dermatophytic fungus classified along with Epidermophyton and Microsporum spp. Dermatophytic fungi are known to cause of dermatophytosis, a cutaneous infection of the skin, hair, and nails, in humans and animals. Trichophyton spp. specifically causes tinea infections such as tinea pedis (Athlete's foot), tinea capitis (ringworms), Tinea crusi (jock itch), and infections of the nails, beards, skin, and scalp. Variants of Trichophyton spp. are spread across the world. There are several species of Trichophyton each with prevalent occurrences and effects depending on transmission and the host of infection.

The transmission of Trichophyton is via direct or indirect contact with an infected individual or through spores. Transmission can be anthropophilic or zoophilic or geophilic. Anthropophilic transmission occurs by direct contact or through fomites, such as contaminated towels, clothing, or shared shower stalls, for example. Zoophilic transmission involves transmission from animals to the human host. Geophilic transmission occurs from soil or infected animals into the human host.

Dermatophytosis caused by Trichophyton can be contracted through contact with infected humans or animals or with contaminated objects, such as towels, bedding, manicure appliances, and hairbrushes. Furthermore, Trichophyton related skin conditions are frequently spread in areas such as pools, spas, locker rooms, wrestling mats, and shared shower facilities.

About 15 million people in the United States currently suffer annually with Trichophyton infections. Physicians commonly recommend an oral fungistat belonging to the flucanozole antifungal agent group for treatment of dermatophytosis caused by Trichophyton infections, which for the most part are ineffective in successfully treating these infections. Often, these fungistats only inhibit and do not kill the underlying infection. Furthermore, the fungistats may affect the kidneys and liver of a person taking such medication. In addition, many over-the-counter and home remedies that claim to treat dermatophytosis also exist, but these treatments have questionable effectiveness. In view of the foregoing, Applicant has identified a need in the art for a treatment that addresses the spread of Trichophyton organisms and that provides a treatment for infections caused by it.

### BRIEF SUMMARY OF THE INVENTION

Embodiments of the presently disclosed invention address the foregoing problems and issues. These and other advantages of the invention, as well as additional inventive features, will be apparent from the description of the invention provided herein.

In particular, embodiments of the disclosure relate to treatments for ringworm on biotic and abiotic surfaces. According to one aspect, a concentrated antimicrobial solution is provided. The concentrated solution includes an antimicrobial composition, a solubilizing agent, and an essential oil. The concentrated antimicrobial solution can be mixed with water, and the resulting solution can be used to disinfect abiotic surfaces from ringworm. According to another aspect, an antimicrobial cream is provided. The antimicrobial cream includes a carrier including a humectant and an antimicrobial composition dispersed in the carrier at 1% w/v or greater. The antimicrobial cream can be applied to biotic surfaces to treat ringworm infections.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 depicts photographs of the first set of PDA plates inoculated with T. rubrum from which the effectiveness in inhibiting the growth of T. rubrum by an antimicrobial composition was determined;

FIG. 2 depicts photographs of the second set of PDA plates inoculated with T. mentagrophytes from which the effectiveness in inhibiting the growth of T. mentagrophytes by an antimicrobial composition was determined; and

FIG. 3 depicts photographs of the PDA plates from which the effectiveness of disinfecting a surface from T. mentagrophytes by an antimicrobial composition was determined.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Various embodiments of a treatment for dermatophytosis (also known as tinea and ringworm) on biotic and abiotic surfaces are provided herein. In general, the treatment includes an antimicrobial composition contained in a suitable vehicle. For example, in treatment of a biotic surface, such as skin, the antimicrobial composition may be incorporated into a suitable lotion or gel. In another example for treatment of an abiotic surface, such as a mat, floor, or shower stall, a concentrated solution of the antimicrobial composition, a solubilizing agent, and an essential oil are added to water such that the treatment solution contains up to about 2% of the antimicrobial solution. The treatment solution is then applied to the abiotic surface to inactivate and inhibit growth of the Trichophyton organisms. These and other aspects and advantages of the presently disclosed treatment will be discussed in relation to the exemplary embodiments provided below.

According to embodiments of the present disclosure, the antimicrobial composition is based on an antimicrobial composition described in U.S. Pat. No. 8,728,462 (granted on May 20, 2014, and owned by the assignee of the present application), the entire contents of which are incorporated herein by reference thereto. The antimicrobial composition of the '462 patent was based on the volatile by-products of Muscodor crispans, absent volatile by-products that are not generally recognized as safe (GRAS), such as naphthalene and azulene. In one or more embodiments, the antimicrobial composition comprises a plurality of compounds selected from C₂-C₅ acids, C₁-C₅ acid esters (i.e., esters of C₁-C₅ alcohols and C₁-C₅ acids), C₂-C₅ alcohols, C₂-C₈ aldehydes, C₂-C₅ ketones, and GRAS volatile by-products of Muscodor crispans.

Example compounds that may be included in an embodiment of the antimicrobial composition include acetaldehyde, benzaldehyde, ethyl acetate, methyl ethyl ketone, methyl isobutyrate, isobutyl alcohol, isoamyl acetate, 2-methylbutyl isobutyrate, isoamyl alcohol, isobutyric acid, phenylethyl acetate, ethanol, isobutyl acetate, isoamyl isobutyrate, isobutyl isovalerate, and isobutyl isobutyrate. In one or more embodiments, a particular composition of the antimicrobial composition includes the following components present in the listed amount: about 0.1 to about 10 wt % of acetaldehyde; about 0.5 to about 25 wt % ethyl acetate, about 0.1 to about 15 wt % of methyl ethyl ketone, about 4 to about 99 wt % of methyl isobutyrate, about 0.1 to about 10 wt % of isobutyl alcohol, about 0.5 to about 25 wt % of isoamyl acetate, about 0.5 to about 25 wt % of 2-methylbutyl isobutyrate, about 2 to about 50 wt % of isoamyl alcohol, about 10 to about 99 wt % of isobutyric acid, about 0.1 to about 10 wt % phenylethyl acetate, about 1.5 to about 40 wt % ethanol, about 0.1 to about 10 wt % of isobutyl acetate, and about 0.1 to about 15 wt % of isobutyl isobutyrate.

According to another example embodiment, the antimicrobial composition includes propanoic acid, one or more C₂-C₅ acid esters, and one or more C₂-C₈ aldehydes. In one or more embodiments, the ratio of propanoic acid to acid ester to aldehyde is 7:2:2 v/v/v. In one or more embodiments, the one or more C₂-C₅ acid esters is selected from ethyl acetate, isoamyl acetate, 2-methylbutyl isobutyrate, isobutyl acetate, isobutyl isovalerate, isoamyl isobutyrate, isobutyl isobutyrate, and combinations thereof. In one or more embodiments, the one or more C₂-C₅ aldehyde is one or both of acetaldehyde and benzaldehyde. According to a preferred embodiment, the antimicrobial composition includes propanoic acid, isobutyl isobutyrate, and benzaldehyde. Advantageously, the foregoing antimicrobial compositions described in this paragraph have a reduced number of components but substantially maintain or enhance the antimicrobial effect of the composition described in the '462 patent.

According to embodiments, the antimicrobial composition (referred to hereinafter as Composition A) includes acetaldehyde, ethyl acetate, methyl ethyl ketone, methyl isobutyrate, isobutyl alcohol, isoamyl acetate, 2-methylbutyl isobutyrate, isoamyl alcohol, isobutyric acid, and phenylethyl acetate. The effectiveness of Composition A in the gas phase inhibition of the growth of Trichophytons was investigated.

In particular, a first set of potato dextrose agar (PDA) plates were inoculated in an X pattern with T. rubrum spores and mycelium, and a second set of PDA plate were inoculated in an X pattern with T. mentagrophytes. In each set of plates, one plate was a control plate containing no Composition A, and the other plate of each set of plates was provided with a plastic well in the center filled with 10 μl of Composition A. The plates were sealed with parafilm wrap and incubated for one week. After one week, the plates were photographed.

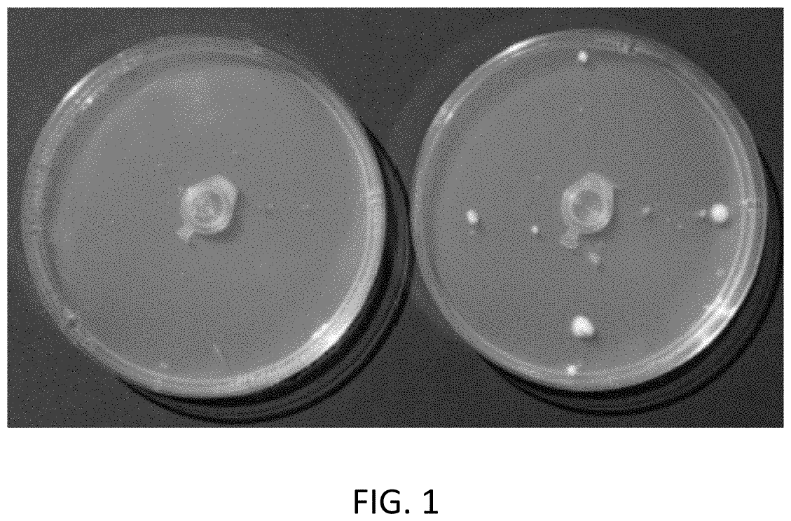

FIG. 1 depicts the first set of PDA plates inoculated with T. rubrum. The plate on the left in FIG. 1 is the plate containing the well of Composition A, and there is substantially no growth of T. rubrum. The plate on the right in FIG. 1 is the control plate, and as can be seen, there is some growth of T. rubrum along the inoculation pattern.

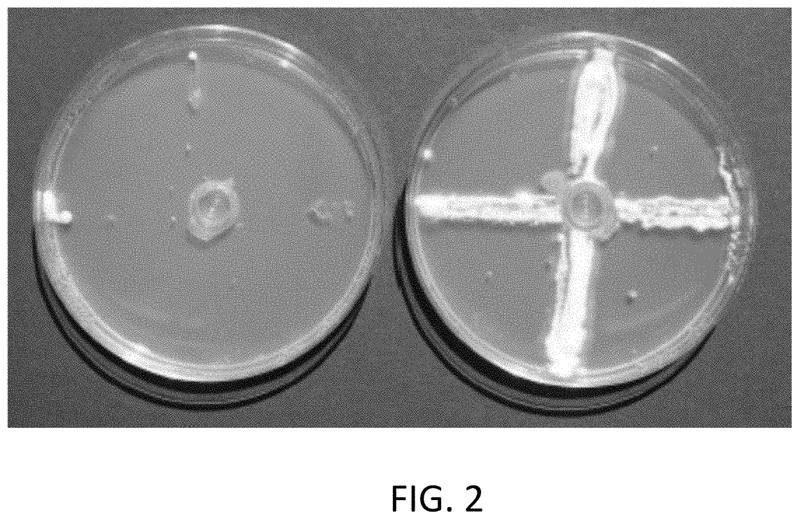

FIG. 2 depicts the second set of PDA plates inoculated with T. mentagrophytes. The plate on the left in FIG. 2 is the plate containing the well of Composition A, and there is very little growth of T. mentagrophytes on the plate. The plate on the right in FIG. 2 is the control plate, and as can be seen, there is significant growth of T. mentagrophytes along the inoculation pattern and even some outside of it. It is noted that T. mentagrophytes is a much faster growing organism that T. rubrum, but Composition A was still effective in inhibiting its growth.

The minimum inhibitory concentration (MIC) was studied to determine the relative response of Trichopytons to Composition A as compared to other fungi. The MIC values were obtained in 1 ml solutions of the George and Camp formula (G&C) which contains per liter 40 g dextrose, 2.5 g casamino acids, 1.8 g of monopotassium phosphate and 0.1 g magnesium sulfate. The fungi were inoculated into the formula as 2×3 mm blocks of agar that were totally infested with the fungus. The blocks had been obtained from a two week old culture of the fungus having been grown on the G&C agar medium (containing 15 g agar per liter). This was done because when the media were inoculated with only the spores and mycelium of the fungus there was no detectable growth even after two weeks. These fungi seem to be thigmotrophic in that they appear to require a three-dimensional target in order to grow. The plates were incubated for 4 days at room temperature and then read for fungal growth emanating from the small agar pieces in the wells. It is also noted that standard potato dextrose broth medium served to support fungal growth as well as the G&C medium. Other fungi were also used in this test for comparative purposes.

The results indicated that the Trichophytons were the most sensitive fungi tested in this regimen of test organisms. Measurements were made at 4 days since the growth was so slow in the case of these organisms. The MICs were the same in both PD broth as well as G&C medium (Table 1). The MIC values were measured as the percent concentration of solution required to inhibit the growth of the pathogen for 48 hours at 23° C.

TABLE 1
MIC measurements using Composition A
and a set of target fungal organisms

| | Standard 1% |
|---|---|
| | Composition A |
| | and dilutions in 1 |
| | ml PD Broth |
| Test Microorganism | (% concentration) |
| Geotrichum candidum | 0.5 |
| Botrytis cinerea | 0.25 |
| Phytophthora cinnamomic | 0.25 |
| Verticillium sp | 0.25 |
| Trichoderma sp | 0.25 |
| Phytophthora palmivora | 0.25 |
| Pythium ultimum | 0.25 |
| Rhizoctonia solani | 0.25 |
| Penicillium sp | 0.25 |
| Saccharomyces sp | 1.0 |
| Bacillus subtilus | 0.25 |
| E. coli | 0.25 |
| C. albicans | 0.5 |
| Botrytis alli | 0.25 |
| Trichophyton rubrum | 0.12 |
| Trichophyton mentagrophytes | 0.06 |

From Table 1, it can be seen that Composition A expresses MIC activities against the two Trichophyton sp, one with an extremely low level of 0.06% (T. mentagrophytes) and the other at 0.12% (T. rubrum).

Additionally, an experiment was performed to determine the effectiveness of Composition A in decontaminating an abiotic surface. In the experiment, one half of the spores and mycelium growing on the surface of a one-week-old G&C plate were physically removed by scraping. The amount removed was equivalent to 34.8 mg of fungal growth, which equated to a total of 204.3±28×10⁵ CFUs of T. mentographphytes. This material was then physically spread over a 10 cm² surface of a previously sterilized surface of black Naugahyde in order to mimic a sporting surface that might be used in wrestling sports or equipment for such activities. The Naugahyde was then incubated for 24 hours in a sealed plastic box with 100% relative humidity.

A 1 liter solution of 20 ml of Composition A, 20 ml ethanol, and remainder of water was prepared. Then the infested surface was generously treated with 5 ml of the solution, and the solution was allowed to stand on the surface for 5 minutes. The excess was wiped clean with a rubberized window wiper. As a control, a sample of infected Naugahyde was treated with sterile water in the same manner as the solution containing Composition A. Thereafter, each Naugahyde surface as was pressed gently onto a PDA plate for 1 minute and removed. The plates were then incubated for 4 days and then photographed.

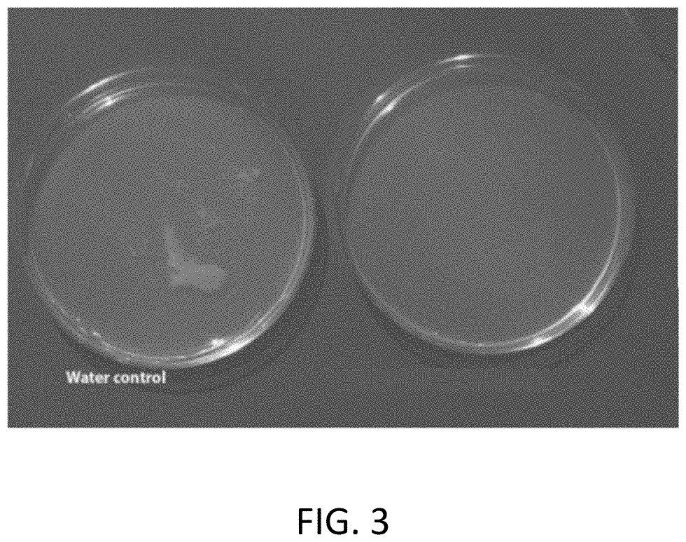

FIG. 3 depicts photographs of the PDA plates. The plate on the left is the control plate, and as can be seen, there is significant fungal growth. The plate on the right is the plate treated with the antimicrobial composition, and there is no visible fungal growth on the plate.

In view of the foregoing, gas assay, MIC, and decontamination experiments, it can be seen that Composition A is effective at inactivating and inhibiting the growth of two of the most common Trichophyton spp. Based on this demonstrated effectiveness, abiotic and biotic treatments were developed that include Composition A.

In one or more embodiments, the biotic treatment includes Composition A incorporated into a topical cream. In such embodiments, the topical cream may comprise a solvent, such as water, and one or more humectants, such as glycerin, panthenol, and aloe vera. In one or more embodiments, the topical cream may further comprise various other compounds, such as emollients, emulsifiers, emulsion stabilizers, antioxidants, preservatives, viscosity controllers, and pH modifiers. Various examples of such compounds include hydrogenated polysiobutene, cetearyl alcohol, ceteareth-20, macadamia nut oil, dimethicone, tocopheryl acetate, stearoxytrimethylsilane, stearyl alcohol, disodium EDTA, sodium hydroxide, potassium sorbate, acrylates/C₁₀-C₃₀ alkyl acrylate crosspolymer, phenoxyethanol, and benzoic acid, amongst others. In one or more embodiments, the amount of Composition A contained in the topical cream is 1% w/v or more and, in embodiments, up to 2% w/v.

The effectiveness of Composition A in treating fungal infections on the skin was determined on volunteer patients. In a first case, twin teenage boys were experiencing ringworm infections on the face, scalp, and neck. They were prescribed an oral fluconazole, which was ineffective at completely eradicating the ringworm infection. For one of the teenage boys, the ringworm sores on his face were treated by directly applying Composition A to the sores. In particular, a 100% solution of Composition A was dabbed on the sores using a cotton swab twice a day over the course of 10 days, during which the sores substantially cleared.

The other teenage boy was treated with a 100% solution of Composition A by dabbing it on the sores of his scalp and neck using a cotton swab twice a day over the course of 3 days. Various sores were treated over the next four to seven days with a diluted solution of Composition A. In particular, a few drops of Composition A were diluted with 10 drops of water, and the diluted solution was applied to the sores. After 7 to 10 days of total treatment, the sores went away. Thereafter, a 1:100 solution of Composition A in water was used as a preventative treatment on the scalp prior to showering to avoid new ringworm infections.

Based on this initial test case, Applicant determined that a Composition A was effective in treating ringworm infections, and Applicant sought to develop a suitable carrier for application of Composition A to the skin.

Thus, in a second test case, Composition A was used to treat a ringworm infection on the neck and shoulder region of a teenage girl. Composition A was formulated with an aloe vera clear cream at a 2% w/v. The girl applied the cream twice daily, and after two weeks, the ringworm skin symptoms substantially disappeared.

In a third case, a middle-aged woman was experiencing a significant ringworm infection on one foot. The ringworm infection was treated with Composition A in an aloe vera clear cream at 2% w/v. The cream was applied nightly for two weeks, and after two weeks, the ringworm infection on the foot had substantially cleared.

It may also be desirable to prevent infections altogether by disinfecting abiotic surfaces from Trichophyton spp. Thus, the present disclosure provides one or more embodiments of a concentrated antimicrobial solution that can be added to water to provide an antimicrobial solution. In embodiments, the concentrated antimicrobial solution includes the antimicrobial composition, a solubilizing agent, and an essential oil. In such embodiments, the concentrated solution includes an antimicrobial composition as described above. Further, in embodiments, the solubilizing agent may be ethanol, and in embodiments, the essential oil is, for example, orange, lavender, clove, spearmint, or eucalyptus oil.

In embodiments, the concentrated antimicrobial solution includes from about 20 vol % to about 35 vol % of the antimicrobial composition, from about 60 vol % to about 80 vol % of the solubilizing agent, and from about 1 vol % to about 5 vol % of the essential oil. In the concentrated antimicrobial solution, the solubilization agent allows for the solubilization of the antimicrobial composition and the essential oil in water. Further, the essential oil allows the antimicrobial composition to reside on the abiotic surface for a longer period of time to not only disinfect microorganisms already present but also any microorganisms that are newly introduced. The essential oil also provides a pleasing fragrance.

In embodiments, the concentrated antimicrobial solution is added to water to create an antimicrobial solution. In embodiments, the antimicrobial solution includes from about 2 vol % to about 10 vol % of the concentrated antimicrobial solution. In one or more embodiments, the antimicrobial solution includes a sufficient amount of the concentrated antimicrobial solution such that at least 2 vol % of the antimicrobial composition is present in the antimicrobial solution.

The antimicrobial solution can be used in a variety of settings to disinfect abiotic surfaces. For example, the antimicrobial solution can be mopped or sprayed onto mats, gym equipment, or shower stalls or used as a foot rinse, and the antimicrobial solution can be used in various school, military, and public settings.

In one test case, an antimicrobial solution containing 1 vol % of the antimicrobial solution was applied to abiotic sporting surfaces in a high school setting. Since application of the antimicrobial solution to the abiotic sporting surfaces, no cases of ringworm have been reported.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A concentrated antimicrobial solution, comprising:
an antimicrobial composition comprising a plurality of compounds selected from C₂-C₅ acids, C₁-C₅ acid esters, C₂-C₅ alcohols, C₂-C₅ aldehydes, and C₂-C₅ ketones;
a solubilizing agent, and
an essential oil.

2. The concentrated antimicrobial solution of claim 1, wherein the solubilizing agent comprises ethanol.

3. The concentrated antimicrobial solution of claim 1, wherein the essential oil comprises at least one of lavender oil, orange oil, spearmint oil, or eucalyptus oil.

4. The concentrated antimicrobial solution of claim 1, comprising from about 20 vol % to about 35 vol % of the antimicrobial composition.

5. The concentrated antimicrobial solution of claim 1, comprising from about 60 vol % to about 80 vol % of the solubilizing agent.

6. The concentrated antimicrobial solution of claim 1, comprising from about 1 vol % to about 5 vol % of the essential oil.

7. The concentrated antimicrobial solution of claim 1, wherein the antimicrobial composition comprises at least one of acetaldehyde, benzaldehyde, ethyl acetate, methyl ethyl ketone, methyl isobutyrate, isobutyl alcohol, isoamyl acetate, 2-methylbutyl isobutyrate, isoamyl alcohol, isobutyric acid, phenylethyl acetate, ethanol, isobutyl acetate, isoamyl isobutyrate, isobutyl isovalerate, or isobutyl isobutyrate.

8. The concentrated antimicrobial solution of claim 1, comprising propanoic acid, one or more C₂-C₅ acid esters, and one or more C₂-C₅ aldehydes.

9. The concentrated antimicrobial solution of claim 8, wherein a ratio of the propanoic acid to the one or more C₂-C₅ acid esters to the one or more C₂-C₈ aldehydes is 7:2:2 v/v/v.

10. An antimicrobial solution, comprising the concentrated antimicrobial solution of claim 1 and water.

11. The antimicrobial solution of claim 10, comprising from about 2 vol % to about 10 vol % of the concentrated antimicrobial solution.

12. The antimicrobial solution of claim 10, comprising at least 2 vol % of the antimicrobial composition.

13. A method of disinfecting an abiotic surface from ringworm, comprising applying the antimicrobial solution of claim 10 to the abiotic surface.

14. The method of claim 13, wherein the abiotic surface comprises at least one of a mat, gym equipment, or a shower.

15. An antimicrobial cream, comprising:
a carrier comprising a humectant;
an antimicrobial composition dispersed in the carrier, the antimicrobial composition comprising a plurality of compounds selected from C₂-C₅ acids, C₁-C₅ acid esters, C₂-C₅ alcohols, C₂-C₈ aldehydes, and C₂-C₅ ketones; and
wherein the antimicrobial composition is dispersed in the carrier at at least 1% w/v.

16. The antimicrobial cream of claim 15, wherein the antimicrobial composition is disperse in the carrier up to 2% w/v.

17. The antimicrobial cream of claim 15, wherein the antimicrobial composition comprises at least one of acetaldehyde, benzaldehyde, ethyl acetate, methyl ethyl ketone, methyl isobutyrate, isobutyl alcohol, isoamyl acetate, 2-methylbutyl isobutyrate, isoamyl alcohol, isobutyric acid, phenylethyl acetate, ethanol, isobutyl acetate, isoamyl isobutyrate, isobutyl isovalerate, or isobutyl isobutyrate.

18. The antimicrobial cream of claim 15, wherein the humectant comprises at least one of glycerin, panthenol, or aloe vera.

19. The antimicrobial cream of claim 15, further comprising water as a solvent for the carrier.

20. A method of treating an infection of ringworm on a biotic surface, comprising applying the antimicrobial cream of claim 15 to the biotic surface.
